(19) United States
(12) Patent Application Publication
(10) Pub. No.: US 2014/0194428 A1
(43) Pub. Date: Jul. 10, 2014

(54) Combination treatments for bipolar disorders

(71) Applicant: Elan Pharmaceuticals. Inc., (US)

(72) Inventors: Susan Abushakra, South San Francisco, CA (US); Gerald Crans, South San Francisco, CA (US); Ramon Hernandez, South San Francisco, CA (US); Jesse Cedarbaum, South San Francisco, CA (US)

(73) Assignee: Elan Pharmaceuticals. Inc., San Francisco, CA (US)

(21) Appl. No.: 13/573,640

(22) Filed: Sep. 28, 2012

(51) Int. Cl. A61K 31/53; A61K 31/19; A61K 31/05

(52) U.S. Cl. CPC A61K 31/53; A61K 31/05; A61K 31/19
USPC 514/242; 514/557

(57) ABSTRACT

The invention relates generally to novel compositions and methods comprising a scyllo-inositol compound and one or both of valproic acid compound and lamotrigine. The compositions and methods provide beneficial effects in the treatment of bipolar disorders.

### FIELD OF THE INVENTION

The invention relates generally to combinations, compositions, conjugates and methods comprising a scyllo-inositol compound and one or both of a valproic acid compound and lamotrigine, and uses thereof.

### BACKGROUND OF THE INVENTION

Bipolar disorder is a brain disorder which causes unusual shifts in a person's mood, thought, energy, behavior, and ability to function. The symptoms of a bipolar disorder can be severe and in some cases result in suicide. Conventional treatments include lithium or valproic acid but they have side effects, and patients do not respond fully, leading to frequent recurrences of mania and depression. Therefore, there remains a need for methods of treating bipolar disorder that provide efficacy while minimizing adverse effects.

### SUMMARY OF THE INVENTION

The invention provides combinations, compositions, conjugates and methods comprising a scyllo-inositol compound, and one or both of a valproic acid compound and lamotrigine. In an aspect, the invention relates to compositions, conjugates, and methods for the prevention, intervention, and/or treatment of a bipolar disorder comprising an effective amount of a scyllo-inositol compound and one or both of a valproic acid compound and lamotrigine.

A combination, composition, conjugate or method comprising a scyllo-inositol compound and one or both of a valproic acid compound and lamotrigine, employing different mechanisms to achieve maximum therapeutic efficacy, improves tolerance to the therapy with a reduced risk of side effects that result from higher doses or longer term monotherapies (i.e., therapies with each compound alone). A composition, conjugate, therapy or method of the invention may permit the use of lower doses of each compound (in particular aspects, lower doses of one or both of a valproic acid compound and lamotrigine) with reduced adverse effects of each compound. A suboptimal dosage provides an increased margin of safety, and also reduces the cost of a drug necessary to achieve prophylaxis and therapy. In addition, in some aspects, a treatment utilizing a single combination dosage unit provides increased convenience and results in enhanced compliance. Other advantages of a combination, composition, conjugate or method disclosed herein includes higher stability towards degradation and metabolism, longer duration of action, and/or longer duration of action or effectiveness at lower doses.

A combination, composition, conjugate or method comprising a scyllo-inositol compound and one or both of a valproic acid compound and lamotrigine reduces, in particular significantly reduces, recurrence of mood episodes or recurrent mood episodes. A composition, conjugate, or method comprising a scyllo-inositol compound and one or both of a valproic acid compound and lamotrigine delays or prevents onset or recurrence of a mood episode. A composition, conjugate, or method comprising a scyllo-inositol compound and one or both of a valproic acid compound and lamotrigine extends, in particular significantly extends, time to onset or recurrence of mood episodes or recurrent mood episodes. A composition, conjugate, or method comprising a scyllo-inositol compound and one or both of a valproic acid compound and lamotrigine delays, in particular significantly delays, time to onset or recurrence of mood episodes.

A combination, composition, conjugate, or method comprising a scyllo-inositol compound and one or both of a valproic acid compound and lamotrigine reduces, in particular significantly reduces recurrence, of depressive episodes or recurrent depressive episodes. A composition, conjugate, or method comprising a scyllo-inositol compound and one or both of a valproic acid compound and lamotrigine delays or prevents onset or recurrence of a depressive episode. A composition, conjugate, or method comprising a scyllo-inositol compound and one or both of a valproic acid compound and lamotrigine extends in particular significantly extend, time to onset or recurrence of depressive episodes or recurrent depressive episodes. A composition, conjugate, or method comprising a scyllo-inositol compound and one or both of a valproic acid compound and lamotrigine may delay, in particular significantly delay, time to onset or recurrence of depressive episodes.

A combination, composition, conjugate, or method comprising a scyllo-inositol compound and one or both of a valproic acid compound and lamotrigine reduces, in particular significantly reduces, recurrence of manic, hypomanic or mixed episodes. A composition, conjugate, or method comprising a scyllo-inositol compound and one or both of a valproic acid compound and lamotrigine delays or prevents onset or recurrence of manic, hypomanic or mixed episodes. A composition, conjugate, or method comprising a scyllo-inositol compound and one or both of a valproic acid compound and lamotrigine extends in particular significantly extends time to onset or recurrence of manic, hypomanic or mixed episodes. A composition, conjugate, or method comprising a scyllo-inositol compound and one or both of a valproic acid compound and lamotrigine delays, in particular significantly delays, time to onset or recurrence of manic, hypomanic or mixed episodes.

A combination, composition, conjugate, or method comprising a scyllo-inositol compound and one or both of a valproic acid compound and lamotrigine reduces, in particular significantly reduces, resistance to conventional treatments. A combination, composition, conjugate, or method comprising a scyllo-inositol compound and one or both of a valproic acid compound and lamotrigine improves, in particular significantly improves, compliance with a conventional treatment.

The invention contemplates a composition, in particular a pharmaceutical composition, comprising a scyllo-inositol compound, and one or both of a valproic acid compound and lamotrigine. The invention also contemplates a pharmaceutical composition comprising an effective amount of a scyllo-inositol compound and an effective amount of one or both of a valproic acid compound and lamotrigine. A pharmaceutical composition optionally comprises a pharmaceutically acceptable carrier, excipient, or vehicle.

In a particular aspect, the invention provides a composition for reducing, in particular significantly reducing recurrence of mood episodes comprising a scyllo-inositol compound, and one or both of a valproic acid compound and lamotrigine. In a particular aspect, the invention provides a composition for reducing, in particular significantly reducing, recurrence of depressive episodes comprising a scyllo-inositol compound, and one or both of a valproic acid compound and lamotrigine. In a particular aspect, the invention provides a composition for reducing, in particular significantly reducing, recurrence of manic, hypomanic or mixed episodes comprising a scyllo-inositol compound, and one or both of a valproic acid compound and lamotrigine.

The invention also contemplates a pharmaceutical composition, comprising a scyllo-inositol compound, and one or both of a valproic acid compound and lamotrigine that provides beneficial effects relative to each compound alone. The beneficial effects provided by a composition of the invention can include enhanced therapeutic effects, in particular sustained therapeutic effects. The beneficial effects provided by a composition of the invention includes the reduction of at least one symptom of a bipolar disorder, or preventing an increase (or slowing the rate of increase) in or delaying the onset of, at least one symptom of a bipolar disorder. Examples of symptoms are disclosed herein and include without limitation, manic episodes, mood episodes, depressive episodes, mixed episodes, hypomania, suicide risk and psychosocial functioning.

The invention provides a method of delaying the occurrence of a mood episode in a subject having bipolar disorder comprising administering to said subject effective amounts of scyllo-inositol and one or both of valproic acid and lamotrigine.

The invention provides a method of reducing the level of myo-inositol in the brain of a subject comprising administering to the subject effective amounts of scyllo-inositol and one or both of valproic acid and lamotrigine.

A beneficial effect may be sustained over several days, weeks, months or years thereby having a major therapeutic impact on the severity of a bipolar disorder and its symptoms. Therefore, the invention also provides a pharmaceutical composition for the treatment of a bipolar disorder comprising an effective amount of a scyllo-inositol compound, and one or both of a valproic acid compound and lamotrigine to provide a sustained beneficial effect following treatment in a pharmaceutically acceptable carrier, excipient, or vehicle. In an aspect, a pharmaceutical composition comprising a scyllo-inositol compound, and one or both of a valproic acid compound and lamotrigine is provided which has been adapted for administration to a subject to provide sustained beneficial effects to treat a bipolar disorder. In an embodiment, the composition is in a form such that administration to a subject results in reduction of recurrence of mood episodes in the subject for a sustained period of time after cessation of treatment. In an embodiment, the composition is in a form such that administration to a subject results in reduction in recurrence of depressive episodes in the subject for a sustained period of time after cessation of treatment. In an embodiment, the composition is in a form such that administration to a subject results in reduction in recurrence of manic, hypomanic or mixed episodes in the subject for a sustained period of time after cessation of treatment.

In an aspect, the invention features a composition comprising a scyllo-inositol compound and one or both of a valproic acid compound and lamotrigine in dosages effective for reducing recurrence or delaying onset of mood episodes, in particular for a sustained period following administration of the composition. In an aspect, the invention features a composition comprising a scyllo-inositol compound and one or both of a valproic acid compound and lamotrigine in dosages effective for reducing recurrence or delaying onset of depressive episodes, in particular for a sustained period following administration of the composition. In an aspect, the invention features a composition comprising a scyllo-inositol compound, and one or both of a valproic acid compound and lamotrigine in dosages effective for reducing recurrence or delaying onset of manic, hypomanic or mixed episodes, in particular for a sustained period following administration of the composition.

A scyllo-inositol compound, and one or both of a valproic acid compound and lamotrigine in a composition or combination of the invention may be in a ratio selected to augment the activity of the valproic acid compound and/or lamotrigine.

Combinations of a scyllo-inositol compound and one or both of a valproic acid compound and lamotrigine in compositions of the invention may be selected to provide unexpectedly additive effects or greater than additive effects i.e. synergistic effects.

In an aspect, the invention provides a pharmaceutical composition comprising a scyllo-inositol compound, and one or both of a valproic acid compound and lamotrigine in combination with a pharmaceutically acceptable carrier, excipient or vehicle, wherein the amounts of a scyllo-inositol compound, and one or both of a valproic acid compound and lamotrigine are selected to provide an additive or synergistic beneficial effect in treating a bipolar disorder.

In an aspect, the invention provides a pharmaceutical composition comprising a scyllo-inositol compound and one or both of a valproic acid compound and lamotrigine in combination, wherein the amounts of scyllo-inositol compound, and one or both of a valproic acid compound and lamotrigine in combination are selected to reduce recurrence or delay time to onset of one or more of mood episodes, depressive episodes, and manic, hypomanic or mixed episodes, as a combined preparation for simultaneous, separate or sequential use in treatment of a bipolar disorder.

In an aspect, the invention provides a pharmaceutical composition comprising a scyllo-inositol compound and one or both of a valproic acid compound and lamotrigine in combination with a pharmaceutically acceptable carrier, excipient or vehicle, wherein the amounts of a scyllo-inositol compound and one or both of a valproic acid compound and lamotrigine are selected to provide an additive effect as a combined preparation for simultaneous, separate or sequential use in treatment of a bipolar disorder.

In an aspect, the invention provides a pharmaceutical composition comprising a scyllo-inositol compound and one or both of a valproic acid compound and lamotrigine in combination with a pharmaceutically acceptable carrier, excipient or vehicle, wherein the amounts of a scyllo-inositol compound and one or both of a valproic acid compound and lamotrigine are selected to provide a synergistic effect as a combined preparation for simultaneous, separate or sequential use in treatment of a bipolar disorder.

In accordance with an aspect, a composition is provided comprising a combination of a scyllo-inositol compound and one or both of a valproic acid compound and lamotrigine effective to exert a synergistic effect in treating a bipolar disorder. In an aspect, the invention provides a pharmaceutical composition comprising an amount of a scyllo-inositol compound and an amount of one or both of a valproic acid compound and lamotrigine wherein said composition achieves a synergistic effect for treating a bipolar disorder in a mammal in need thereof.

The invention provides a synergistic composition comprising sufficient amounts of a scyllo-inositol compound and one or both of a valproic acid compound and lamotrigine to achieve a desired result that is greater than the result achieved with each component on its own. In an aspect, the invention provides a method of treating bipolar disorder in a subject in need thereof comprising the step of administering to the subject an effective amount of a synergistic composition comprising a scyllo-inositol compound and one or both of a valproic acid compound and lamotrigine.

In an aspect, the invention provides a maintenance therapy for bipolar disorder in a subject comprising administration of a scyllo-inositol compound and one or both of a valproic acid compound and lamotrigine, or a composition or conjugate of the invention to the subject.

In an aspect, the invention provides improving compliance with a conventional treatment for a bipolar disorder comprising administering to a subject receiving a conventional treatment a scyllo-inositol compound. In an embodiment the conventional treatment is lithium. In an embodiment the conventional treatment is a valproic acid compound. In an embodiment the conventional treatment is lamotrigine.

In particular aspects, a composition, in particular a synergistic composition, comprises scyllo-inositol and one or both of valproic acid and lamotrigine, one or more of which are in doses that are at least 1 to 5, 1 to 10 fold, 2 of 5, 2 to 10 fold, or 5 to 10 fold lower, in particular 2, 3 or 4 fold lower, than the doses of each component required to treat a bipolar disorder alone.

The invention provides a pharmaceutical composition in separate containers and intended for simultaneous or sequential administration to a subject, comprising a scyllo-inositol compound, and one or both of a valproic acid compound and lamotrigine, both optionally together with pharmaceutically acceptable carriers, excipients, or vehicles.

The invention provides a conjugate comprising a scyllo-inositol compound, interacting with or linked to a valproic acid compound or lamotrigine.

The invention also provides methods for preparing compositions and conjugates of the invention. In an aspect, the invention provides a method of preparing a pharmaceutical composition comprising a scyllo-inositol compound and one or both of a valproic acid compound and lamotrigine. A method can comprise mixing a scyllo-inositol compound and one or both of a valproic acid compound and lamotrigine, and a pharmaceutically acceptable carrier, excipient, or vehicle, in particular, a pharmaceutically acceptable carrier, excipient, or vehicle effective to stabilize the scyllo-inositol compound and one or both of a valproic acid compound and lamotrigine. After compositions have been prepared, they can be placed in an appropriate container(s) and labeled for treatment of an indicated condition. For administration of a composition of the invention, such labeling would include amount, frequency and method of administration. In another aspect the invention provides a method of preparing a stable pharmaceutical composition of a scyllo-inositol compound and one or both of a valproic acid compound and lamotrigine comprising preparing a composition comprising the scyllo-inositol compound and one or both of a valproic acid compound and lamotrigine and a pharmaceutically acceptable carrier, excipient, or vehicle effective to stabilize the scyllo-inositol compound and one or both of a valproic acid compound and lamotrigine.

The invention relates to the treatment of a dipolar disorder in a subject using a scyllo-inositol compound and one or both of a valproic acid compound and lamotrigine, a composition, combination treatment, and/or conjugate of the invention. In an aspect, the invention provides a method for treating a bipolar disorder in a subject comprising administering to the subject an effective amount of a scyllo-inositol compound and one or both of a valproic acid compound and lamotrigine. In an aspect, the invention provides a method for treating a bipolar disorder in a subject comprising co-administering a scyllo-inositol compound and one or both of a valproic acid compound and lamotrigine, or a composition of the invention to a subject in need thereof. Another aspect of this invention is a method of treating a bipolar disorder comprising administering to a patient, either together or separately, a scyllo-inositol compound and one or both of a valproic acid compound and lamotrigine. In an aspect the invention provides a treatment which results in sustained beneficial effects following treatment.

In an aspect, the invention provides a method of treating a patient suffering from a bipolar disorder non-responsive to a conventional treatment, comprising administering to the patient a therapeutically effective amount of a scyllo-inositol compound and optionally one or both of a valproic acid compound and lamotrigine. In an embodiment, the conventional treatment is lithium.

In an aspect, the invention provides a method of treating a patient suffering from a bipolar disorder who does not respond to or who cannot tolerate valproate therapy alone comprising administering to the patient a therapeutically effective amount of a scyllo-inositol compound.

In an aspect, the invention provides a method of treating a patient suffering from a bipolar disorder who does not respond to or who cannot tolerate lithium therapy comprising administering to the patient a therapeutically effective amount of a scyllo-inositol compound and one or both of a valproic acid compound and lamotrigine.

In an aspect the invention provides a method of reducing resistance to treatment with a conventional treatment comprising administering to the patient a therapeutically effective amount of a scyllo-inositol compound and optionally one or both of a valproic acid compound and lamotrigine.

In an aspect, the invention provides a method of treating a patient suffering from a bipolar disorder that has had a sub-optimal response to treatment with a conventional treatment, comprising administering to the patient an effective amount of a scyllo-inositol compound and optionally one or both of a valproic acid compound and lamotrigine, to provide an enhanced or optimal response.

The invention contemplates a method of retreatment using a therapeutically effective amount of a scyllo-inositol compound and optionally one or both of a valproic acid compound and lamotrigine, for patients suffering from a bipolar disorder who fail to respond to a conventional treatment, or who following cessation of such treatment suffered a relapse or who relapse while on treatment.

The invention provides a combination treatment for a bipolar disorder comprising administering to a subject in need thereof effective amounts of a scyllo-inositol compound and one or both of a valproic acid compound and lamotrigine. In an aspect the invention provides a combination treatment providing synergistic activity or delivering synergistically effective amounts of a scyllo-inositol compound and one or both of a valproic acid compound and lamotrigine.

In embodiments, the compounds, compositions, and/or conjugates of the invention are administered orally or systemically.

In a further aspect, the invention provides a method for ameliorating progression of a bipolar disorder or obtaining a less severe stage of a bipolar disorder in a subject suffering from a bipolar disorder comprising administering an effective amount of a scyllo-inositol compound and one or both of a valproic acid compound and lamotrigine, or a composition of the invention.

The invention relates to a method of delaying the progression of a bipolar disorder comprising administering an effective amount a scyllo-inositol compound and one or both of a valproic acid compound and lamotrigine, or a composition or conjugate of the invention.

The invention provides a method of treating a bipolar disorder comprising administering a scyllo-inositol compound and one or both of a valproic acid compound and lamotrigine in effective amounts for reducing at least one symptom of a bipolar disorder or for reducing, preventing an increase (or slowing the rate of increase) in or delaying the onset of, at least one of symptom of a bipolar disorder. In an aspect, the invention provides a method of treating an individual exhibiting one or more symptoms of bipolar disorder, the method comprising administering to the individual a therapeutically-effective amount of a scyllo-inositol compound and one or both of a valproic acid compound and lamotrigine. In embodiments, the symptoms comprise mood episodes, depressive episodes, manic episodes, mixed episodes, hypomania, suicide risk and psychosocial functioning.

The invention provides a method for reducing, in particular significantly reducing, recurrence or delaying onset or recurrence of mood episodes in a subject suffering from a bipolar disorder comprising administering to the subject an effective amount of a scyllo-inositol compound and one or both of a valproic acid compound and lamotrigine. In a particular aspect, the invention provides a method for reducing, in particular significantly reducing, recurrence, or delaying, in particular significantly delaying, onset or recurrence of depressive episodes in a subject suffering from a bipolar disorder comprising administering to the subject an effective amount of a scyllo-inositol compound and one or both of a valproic acid compound and lamotrigine. In a particular aspect, the invention provides a method for reducing, in particular significantly reducing, recurrence, or delaying, in particular significantly delaying, onset or recurrence of manic, hypomanic or mixed episodes in a subject suffering from a bipolar disorder comprising administering to the subject an effective amount of a scyllo-inositol compound and one or both of a valproic acid compound and lamotrigine.

An embodiment of the invention provides a method for treating a bipolar disorder comprising administering to a mammal in need thereof a combination of a scyllo-inositol compound and one or both of a valproic acid compound and lamotrigine in an amount sufficient to inhibit or reduce mania, mixed episodes, hypomania, suicide risk or psychosocial functioning thereby treating the bipolar disorder.

In an aspect, the invention provides a therapeutic method which comprises identifying a patient diagnosed with a bipolar disorder and treating the patient with an effective amount of a scyllo-inositol compound and one or both of a valproic acid compound and lamotrigine. A patient may be diagnosed with a bipolar disorder or a bipolar disorder may be characterized using methods known in the art, for example using the Mood Disorders Questionnaire, the Bipolar Spectrum Diagnostic Scale, the Structural Clinical Interview for DSM Disorders (SCID), the DSM-IV-TR (2004) Criteria for Diagnosis of Borderline Personality Disorder, the Montgomery-Åsberg Depression Rating Scale (MADRS), the Young Mania Rating Scale (YMRS), the Columbia-Suicide Severity Rating Scale (C-SSRS) and/or the Mood Swings Survey (MSS).

In an aspect, the invention provides a therapeutic method which comprises identifying a patient suffering from a bipolar disorder that is non-responsive to conventional treatment, and treating the patient with an effective amount of a scyllo-inositol compound and optionally one or both of a valproic acid compound and lamotrigine.

The invention has particular applications in treating bipolar I disorder. In an aspect, the invention provides a method for treating bipolar I disorder comprising administering an effective amount of a scyllo-inositol compound and one or both of a valproic acid compound and lamotrigine, or a composition or conjugate of the invention

The invention also contemplates the use of a scyllo-inositol compound and one or both of a valproic acid compound and lamotrigine, a composition, a conjugate, or combination treatment of the invention for treating a bipolar disorder. Further, the invention relates to the use of a scyllo-inositol compound and one or both of a valproic acid compound and lamotrigine, a pharmaceutical composition or a conjugate disclosed herein for the preparation of a medicament for treating a bipolar disorder. In an embodiment, the invention relates to the use of synergistically effective amounts of a scyllo-inositol compound and one or both of a valproic acid compound and lamotrigine, for the preparation of a medicament for treating a bipolar disorder.

Since the present invention in part relates to a method of treatment comprising a combination of active agents which may be administered separately or as conjugates, the invention also provides a kit comprising a scyllo-inositol compound and one or both of a valproic acid compound and lamotrigine, a pharmaceutical composition, or conjugate of the invention in kit form. In an aspect, the invention provides a kit comprising a scyllo-inositol compound and one or both of a valproic acid compound and lamotrigine, or a pharmaceutical composition of the invention. In particular, the invention provides a kit for treating bipolar I disorder, containing a composition comprising a scyllo-inositol compound and one or both of a valproic acid compound and lamotrigine, a container, and instructions for use. The composition of the kit can further comprise a pharmaceutically acceptable carrier, excipient, or vehicle.

These and other aspects, features, and advantages of the present invention should be apparent to those skilled in the art from the following detailed description.

### DETAILED DESCRIPTION OF THE INVENTION

Unless defined otherwise, all technical and scientific terms used herein have the same meaning as commonly understood by one of ordinary skill in the art to which this invention belongs.

Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.” The term “about” means plus or minus 0.1 to 50%, 5-50%, or 10-40%, for example 10-20%, for example 10% or 15%, of the number to which reference is being made. Further, it is to be understood that “a,” “an,” and “the” include plural referents unless the content clearly dictates otherwise.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

An “additive effect” of a scyllo-inositol compound and one or both of a valproic acid compound and lamotrigine refers to an effect that is equal to the sum of the effects of the individual compounds.

The term “administering” or “administration” refers to the process by which a scyllo-inositol compound and one or both of a valproic acid compound and lamotrigine, compositions and/or conjugates disclosed herein are delivered to a subject for treatment. A scyllo-inositol compound and one or both of a valproic acid compound and lamotrigine, compositions and/or conjugates are administered in accordance with good medical practices taking into account the subject's clinical condition, the site and method of administration, dosage, subject age, sex, body weight, and other factors known to the physician.

The terms “associated”, “linked”, “interact”, “interaction”, or “interacting” refer to any physical association between molecules. In an embodiment, the terms refer to a stable association between two molecules due to, for example, electrostatic, hydrophobic, ionic, hydrogen-bond interactions, or covalent interactions.

A “beneficial effect” refers to an effect of a scyllo-inositol compound and one or both of a valproic acid compound and lamotrigine, composition or conjugate disclosed herein, in certain aspects of the invention, including favorable or enhanced pharmacological and/or therapeutic effects, and/or improved biological activity. In aspects of the invention, the beneficial effects include without limitation reduced or delayed manic, hypomanic, depressive or mixed episodes or recurrence of such episodes, and reduced suicide risk, and/or psychosocial functioning.

In an embodiment, the beneficial effect is a “sustained beneficial effect” where the beneficial effect is sustained for a prolonged period of time after termination of treatment. A beneficial effect can be sustained over several days, weeks, months or years thereby having a major beneficial impact on the severity of a bipolar disorder. In aspects of the invention, a beneficial effect is sustained for a prolonged period of at least about 2 to 4 weeks, 2 to 5 weeks, 3 to 5 weeks, 2 to 6 weeks, 2 to 8 weeks, 2 to 10 weeks, 2 to 12 weeks, 2 to 14 weeks, 2 to 16 weeks, 2 to 20 weeks, 2 to 24 weeks, 2 weeks to 12 months, 2 weeks to 18 months, 2 weeks to 24 months, or several years following treatment. The period of time a beneficial effect is sustained may correlate with the duration and timing of the treatment. A subject may be treated continuously for about or at least about 2 to 4 weeks, 2 to 6 weeks, 2 to 8 weeks, 2 to 10 weeks, 2 to 12 weeks, 2 to 14 weeks, 2 to 16 weeks, 2 weeks to 6 months, 2 weeks to 12 months, 2 weeks to 18 months, or several years, periodically or continuously.

The beneficial effect may be a statistically significant effect in terms of statistical analysis of an effect of a scyllo-inositol compound and one or both of a valproic acid compound and lamotrigine, versus the effects without the compounds or with each individual compounds. “Statistically significant” effects may represent levels that are higher or lower than a standard. In embodiments of the invention, the difference may be 1.5, 2, 3, 4, 5, 6, 7, 8, 9, 10, 15, 20, 30 40, or 50 times higher or lower compared with the effect obtained without a scyllo-inositol compound and one or both of a valproic acid compound and lamotrigine or with an individual compound.

“Bipolar disorder” is a psychiatric disorder characterized by dramatic mood swings, from episodes of mania to depression, often with periods of normal moods in between. A bipolar disorder is also associated with increased cardiovascular morbidity or suicide risk. A bipolar disorder includes, bipolar I disorder, bipolar II disorder, rapid-cycling bipolar disorder, cyclothymia, acute mania, drug-induced mania, or drug-induced hypomania, and other types of depressive and mood disorders well known by those of skill in the art.

A bipolar disorder can be diagnosed following the guidelines in the Diagnostic and Statistical Manual of Mental Disorders (DSM) Fourth Edition, 1994 (American Psychiatric Association, 1400 K Street NW, Suite 1101, Washington, D.C. 20005-2403 USA). Tools which may be utilized in diagnosing a bipolar disorder include, without limitation, the Mood Disorders Questionnaire (see, for example, Twiss J et al, J Affect Disord. 2008 Sep; 110 (1-2):180-4 and references therein), the Bipolar Spectrum Diagnostic Scale (see, for example, Nassir Ghaemi S, et al, J Affect Disord. 2005 Feb; 84 (2-3):273-7 and references therein), the Structural Clinical Interview for DSM Disorders (SCID) [see for example, Dreessen L, et al, J Personal Disord 1998 Summer; 12 (2):149-61 and Spitzer R L, et al, Arch Gen Psychiatry 1992 Aug; 49 (8):624-9 and references therein], the DSM-IV-TR (2004) Criteria for Diagnosis of Borderline Personality Disorder (see for example, Lieberman D Z et al, Psychopathology, 2008; 41 (1):35-8 and references therein), the Montgomery-Åsberg Depression Rating Scale (MADRS) [see for example, Test: Montgomery-Åsberg Depression Rating Scale (MADRS) in BioPsychoSocial Assessment Tools for the Elderly-Assessment Summary Sheet. The University of Western Ontario, London, Ontario, Canada. In turn citing: Müller-Thomsen T, Arlt S, Mann U, Maβ R, Ganzer S. (2005), Archives of Clinical Neuropsychology, 20:271-6, and McDowell, I. (2006). Measuring Health: A guide to rating scales and questionnaires 3rd Ed. New York: Oxford University Press], the Young Mania Rating Scale (YMRS) (see for example, Young R C, Biggs J T, Ziegler V E, Meyer D A. Young Mania Rating Scale. In: Handbook of Psychiatric Measures. Washington, D.C.: American Psychiatric Association; 2000:540-542), the Columbia-Suicide Severity Rating Scale (C-SSRS) [see for example, Posner K, Am J Psychiatry, 2011 Dec; 168 (12):1266-77 and references therein] and/or the Mood Swings Survey (MSS) (see, for example, Parker G et al, J Affect Disord. 2008 Jul; 109 (1-2):83-9 and the references therein).

Bipolar I disorder is generally characterized by the occurrence of one or more manic episodes or mixed episodes, and often one or more major depressive episodes. Bipolar II disorder is generally characterized by the occurrence of one or more major depressive episodes accompanied by at least one hypomanic episode. Rapid-cycling bipolar disorder occurs when four or more episodes of illness occur within a 12 month period in a patient. Cyclothymia is a mild form of bipolar disorder in which an individual has mood swings over a period of years that go from mild depressive episodes to euphoria and excitement.

A manic episode as defined by criteria set forth in the DSM-IV, includes the following symptoms: inflated self-esteem or grandiosity, increased energy, activity, and restlessness; excessively high, overly good, euphoric mood; extreme irritability; racing thoughts and talking very fast, jumping from one idea to another; distractibility; decreased need for sleep; unrealistic belief in one's abilities and powers; poor judgment; excessive involvement in pleasurable activities that have a high potential for painful consequences (e.g. engaging in spending sprees, sexual indiscretions or foolish business investments); a lasting period of behavior that is different from usual; increased sexual drive; abuse of drugs (particularly cocaine, alcohol, and sleeping medications); and provocative, intrusive, or aggressive behavior. Diagnosis of a manic episode may be made if elevated mood occurs with three or more of the other symptoms most of the day, nearly every day, or for one week or longer. In particular, diagnosis of a manic episode may be made when there is a distinct period of abnormally and persistently elevated, expansive, or irritable mood, lasting at least 1 week or any duration if the individual is hospitalized.

A depressive episode as defined by criteria set forth in the DSM-IV, includes the following symptoms: lasting sad, anxious, or empty mood; feelings of hopelessness or pessimism; feelings of excessive or inappropriate guilt; feelings of worthlessness, or helplessness; loss of interest or pleasure in activities once enjoyed; decreased energy, fatigue or loss of energy nearly every day; diminished ability to concentrate, remember, or make decisions; restlessness or irritability; insomnia or hypersomnia nearly every day; significant weight loss when not dieting or weight gain or decrease or increase in appetite nearly every day; chronic pain or other persistent bodily symptoms that are not caused by physical illness or injury; recurrent thoughts of death; and recurrent suicidal thoughts, suicide attempts or a specific plan for committing suicide. Diagnosis of a depressive episode may be made if five or more of these symptoms last most of the day, nearly every day, or for a period of two weeks or longer. In particular a diagnosis of a depressive episode may be made if five or more of the symptoms have been present during the same 2-week period and represent a change from previous functioning and at least one of the symptoms is either depressed mood or loss of interest or pleasure.

A mixed episode meets the criteria for both a manic episode or depressive episode as defined in the DSM-IV, except for the duration, with symptoms occurring nearly every day during at least a one-week period. Symptoms of a mixed episode often include agitation, trouble sleeping, a significant change in appetite, psychosis, and suicidal thinking. An individual may be very sad and feel hopeless but at the same time feel extremely energized.

A hypomanic episode is a mild to moderate level of mania, and as defined in the DSM-IV, includes the following symptoms: inflated self-esteem or grandiosity, decreased need for sleep; more talkative than usual or pressure to keep talking; racing thoughts; distractibility, increase in goal-directed activity or psychomotor agitation; and excessive involvement in pleasurable activities that have a high potential for painful consequences (e.g. engaging in spending sprees, sexual indiscretions or foolish business investments). A hypomanic episode is associated with an unmistakable change in functioning that is uncharacteristic of the person when not symptomatic. The episode may feel good to the individual and may even be associated with good functioning and enhanced productivity. A hypomanic episode has no psychotic features. Hypomania can become severe mania or switch into depression if not treated properly.

Severe manic or depressive episodes include symptoms of psychosis including without limitation hallucinations and delusions. The psychotic symptoms often reflect the extreme mood state of the individual at the time. For example, delusions of grandiosity may occur during mania while delusions of guilt or worthlessness may appear during depression.

In an embodiment, the bipolar disorder is acute bipolar disorder.

In a particular embodiment, the bipolar disorder is bipolar I disorder.

In a particular embodiment, the bipolar disorder is bipolar II disorder.

In a particular embodiment, the bipolar disorder is mixed bipolar disorder.

In a particular embodiment, the bipolar disorder is rapid-cycling bipolar disorder.

In a particular embodiment, the bipolar disorder is hypomania, cyclothymia, acute mania, drug-induced mania, or drug-induced hypomania.

A “combination treatment” and “in combination” mean that the active ingredients are administered concurrently to a patient being treated. When administered in combination each component may be administered at the same time, or sequentially in any order at different points in time. Therefore, each component may be administered separately, but sufficiently close in time to provide the desired effect, in particular a beneficial, additive, or synergistic effect. The first compound may be administered in a regimen that additionally comprises treatment with another compound. In aspects the terms refer to the administration of a scyllo-inositol compound and one or both of a valproic acid compound and lamotrigine, including separate administration of medicaments each containing one of the compounds as well as simultaneous administration whether or not the compounds are combined in one formulation or whether they are in separate formulations.

A “conventional treatment” includes approved treatments for bipolar disorders, such as lithium, valproate, divalproex, quetiapine, risperidone, olanzapine, aripiprazole, lamotrigine, asenapine, arisulpride, ziprasidone, carbamazepine, oxcarbazepine, paliperidone, fluoxetine, benzodiazepine or combinations thereof. In aspects of the invention, the conventional treatment is chosen from or selected from the group consisting of lithium, valproate, divalproex and lamotrigine, or combinations thereof. In particular aspects, the conventional treatment is lithium. In particular aspects, the conventional treatment is valproate.

An “effective amount” or “therapeutically effective amount” of a compound (i.e., scyllo-inositol, valproate compound or lamotrigine), means the amount or dose of the compound that provides the desired treatment or prophylactic effects in a patient, for example, reducing the severity or frequency of occurrence of a symptom of a bipolar disorder. An effective amount of a compound can vary according to factors such as the particular type of disorder, the mode and route of administration; the species, age, sex, health, medical condition, and weight of the patient, the nature and extent of the symptoms, the kind of concurrent treatment, the frequency of treatment, the renal and hepatic function of the patient, and the desired effect. A dosage regimen may be adjusted to provide the optimum therapeutic response. For example, several divided doses may be administered daily or the dose may be proportionally reduced as indicated by the exigencies of the therapeutic situation.

Lamotrigine, 6-(2,3-dichlorophenyl)-1,2,4-triazine-3,5-diamine), is an anticonvulsant and mood stabilizing drug that has been used in the treatment of bipolar disorder and epilepsy. Lamotrigine is commercially available under the brand names Lamictal™, Lamotrigine™, Lamotrine™, Lamitrin™, Lamictin™, Lamogine™, and Lamitor™. The drug is available from various generic companies including Teva, Taro, Dr. Reddy's Laboratories Ltd., Watson Pharmaceuticals and Zydus Pharmaceuticals USA Inc.

A “maintenance therapy” refers to a therapy that is designed to manage a disorder or to help a primary treatment succeed. A maintenance therapy may be given to a subject to prevent a recurrence or relapse or adverse outcome. A maintenance therapy may be given to a subject suffering from a bipolar disorder to reduce residual symptoms, delay and prevent recurrence of new episodes, reduce the risk of suicide and/or enhance psychosocial functioning.

The term “pharmaceutically acceptable carrier, excipient, or vehicle” refers to a medium which does not interfere with the effectiveness or activity of an active ingredient and which is not toxic to the hosts to which it is administered. A carrier, excipient, or vehicle includes diluents, binders, adhesives, lubricants, disintegrates, bulking agents, wetting or emulsifying agents, pH buffering agents, and miscellaneous materials such as absorbents that may be needed in order to prepare a particular composition. Examples of carriers, excipients, and vehicles include but are not limited to saline, buffered saline, dextrose, water, glycerol, ethanol, and combinations thereof. The use of such media and agents for an active substance is well known in the art. Acceptable carriers, excipients or vehicles may be selected from any of those commercially used in the art, in particular, those used in pharmaceutical compositions of a scyllo-inositol compound and one or both of a valproic acid compound and lamotrigine.

The terms “subject”, “individual”, or “patient” refer to an animal including a warm-blooded animal such as a mammal, which is afflicted with or suspected of having or being pre-disposed to a bipolar disorder. Mammal includes without limitation any members of the Mammalia. In general, the terms refer to a human. Typical subjects for treatment include persons susceptible to, suffering from or that have suffered a bipolar disorder. In embodiments of the invention the subjects are susceptible to, or suffer from bipolar I disorder.

The term “treating” refers to reversing, alleviating, or inhibiting the progress of a bipolar disorder, or one or more symptoms of a bipolar disorder. The term also refers to reducing the severity of a bipolar disorder or symptoms associated with a bipolar disorder prior to or after affliction with the disorder. Reduction of the severity of a bipolar disorder prior to affliction refers to administration of compounds, a composition or conjugate disclosed herein to a subject that at the time of administration does not have symptoms or detectable symptoms of a bipolar disorder. Depending on the condition of the subject, the term also refers to preventing a bipolar disorder, and includes preventing the onset, or preventing the symptoms associated with a bipolar disorder. “Preventing” also refers to preventing the recurrence of a bipolar disorder, or recurrence of one or more symptoms associated with a bipolar disorder. The terms “treatment” and “therapeutically,” refer to the act of treating, as “treating” is defined above. The purpose of prevention and intervention is to combat the disorder and includes the administration of the active compounds to prevent or delay the onset or recurrence of the symptoms or complications, or alleviating the symptoms or complications, or eliminating the disorder.

A “scyllo-inositol compound” includes compounds having the structure of the formula Ia or Ib:

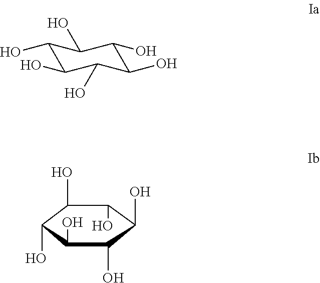

A scyllo-inositol compound includes a compound of the formula Ia or Ib wherein one to six, one to five, one, two, three or four, hydroxyl groups, are replaced by substituents, in particular univalent substituents, with retention of configuration and activity (e.g., reduces the level of myo-inositol in a patient's brain). Suitable substituents include without limitation hydrogen; alkyl; substituted alkyl; acyl; alkenyl; substituted alkenyl; alkynyl; substituted alkynyl; alkoxy; substituted alkoxy; halogen; thiol; —NHR¹ wherein R¹ is hydrogen, acyl, alkyl or —R²R³ wherein R² and R³ are the same or different and represent acyl or alkyl; —SR⁴ wherein R⁴ is hydrogen, alkyl, or —O₃H; or —OR⁵ wherein R⁵ is alkyl or —SO₃H.

Particular aspects of the invention utilize scyllo-inositol compounds of the formula Ia or Ib wherein one or more of the hydroxyl groups is replaced with alkyl, in particular C₁-C₄ alkyl, more particularly methyl; acyl; chloro or fluoro; alkenyl; —NHR¹ wherein R¹ is hydrogen, acyl, alkyl or —R²R³ wherein R² and R³ are the same or different and represent acyl or alkyl; —SR⁴ wherein R⁴ is hydrogen, alkyl, or —O₃H; and —OR⁵ wherein R⁵ alkyl, or —SO₃H.

Particular aspects of the invention utilize scyllo-inositol compounds of the formula Ia or Ib wherein one of the hydroxyl groups is replaced with alkyl, in particular C₁-C₄ alkyl, more particularly methyl or ethyl.

Particular aspects of the invention utilize scyllo-inositol compounds of the formula Ia or Ib wherein one of the hydroxyl groups is replaced with halogen, in particular chloro or fluoro.

Particular aspects of the invention utilize scyllo-inositol compounds of the formula Ia or Ib wherein one of the hydroxyl groups is replaced with thiol.

In embodiments of the invention, the scyllo-inositol compound is scyllo-inositol. In particular embodiments the scyllo-inositiol compound is AZD-103/ELND005 from Elan Corporation (Ireland).

Scyllo-inositol compounds may be prepared using conventional processes or they may be obtained from commercial sources. For example, scyllo-inositol compounds can be prepared using chemical and/or microbial processes. In aspects of the invention, scyllo-inositol is produced using process steps described by M. Sarmah and Shashidhar, M., Carbohydrate Research, 2003, 338, 999-100, Husson, C., et al, Carbohydrate Research 307 (1998) 163-165; Anderson R. and E. S. Wallis, J. American Chemical Society (US), 1948, 70:2931-2935; Weissbach, A., J Org Chem (US), 1958, 23:329-330; Chung, S. K. et al., Bioorg Med Chem. 1999, 7 (11):2577-89; or Kiely D. E., and Fletcher, H. G., J. American Chemical Society (US) 1968, 90:3289-3290; described in JP09-140388, DE 3,405,663 (Merck Patent GMBH), JP04-126075, JP05-192163, or WO06109479, or described in WO0503577, US20060240534, EP1674578, JP9140388, JP09140388, JP02-184912, JP03-102492 (Hokko Chemical Industries). In a particular embodiment, scyllo-inositol is prepared according to the processes described in WO2011100670. In particular aspects of the invention, scyllo-inositol is prepared using the chemical process steps described in Husson, C., et al, Carbohydrate Research 307 (1998) 163-165. In other aspects of the compositions, conjugates, methods and uses disclosed herein, scyllo-inositol is prepared using microbial process steps similar to those described in WO05035774 (EP1674578 and US20060240534) JP2003102492, or JP09140388 (Hokko Chemical Industries). Derivatives may be produced by introducing substituents into scyllo-inositol using methods well known to a person of ordinary skill in the art.

“Suboptimal dose” or suboptimal dosage” refers to a dose or dosage of an active compound which is less than the optimal dose or dosage for that compound when used in monotherapy.

A “synergistic effect” refers to an effect that is greater than the additive effect which results from the sum of the effects of the individual compounds. A synergistic effect can work through similar or different mechanisms or pathways of action. One potential advantage of a combination therapy with a synergistic effect is that standard dosages may be used for a greater therapeutic effect than expected from the addition of the effect of each compound administered alone; or alternatively lower dosages or reduced frequency of administration of the therapeutic compound(s) may be used to achieve a better therapeutic effect.

A “valproic acid compound” includes valproic acid (2-propylpentanoic acid) or divalproex, which is an anticonvulsant and mood-stabilizing drug that has been used in the treatment of bipolar disorder, as well as to treat migraine headaches, epilepsy and schizophrenia. Valproic acid is a liquid at room temperature, but it can be reacted with a base to form a solid salt such as sodium valproate or divalproex sodium. A valproic acid compound includes, without limitation, valproic acid or divalproex, salts of valproic acid or divalproex (e.g., sodium valproate or divalproex sodium), and mixtures of the acid and salts (e.g., valproate semisodium). A valproic acid compound can be in the form of a capsule, an extended-release tablet, a delayed-release tablet, a sprinkle capsule or a syrup. The syrup, capsules, delayed-release tablets and sprinkle capsules are usually taken two or more times daily. The extended-release tablets are usually taken once a day. Valproic acid compounds are commercially available under the various brand names Depakote™, Depakote ER™, Depakene™, Depakene Crono™, Depacon™, Depakine™, Valparin™ and Stavzor™.

In general “pure” means better than 90%, 92%, 94%, 95%, 98% or 99% pure, and “substantially pure” means a compound synthesized such that the compound, as made available for consideration into a therapeutic dosage, has only those impurities that can not readily nor reasonably be removed by conventional purification processes. A scyllo-inositol compound and one or both of a valproic acid compound and lamotrigine may be pure or substantially pure.

### Compositions and Conjugates

A scyllo-inositol compound and one or both of a valproic acid compound and lamotrigine may be formulated into a pharmaceutical composition for administration to a subject. A pharmaceutical composition may be a formulation including without limitation pills, tablets, caplets, soft and hard gelatin capsules, sprinkles, lozenges, sachets, cachets, vegicaps, liquid drops, elixirs, suspensions, emulsions, solutions, syrups, aerosols (as a solid or in a liquid medium) suppositories, sterile injectable solutions, sustained release formulations, and/or sterile packaged powders, which comprise a scyllo-inositol compound and one or both of a valproic acid compound and lamotrigine. Various delivery systems are known and can be used to administer a composition of the invention, e.g. encapsulation in liposomes, microparticles, microcapsules, and the like. Compositions of the invention can be formulated as pharmaceutically acceptable salts.

Pharmaceutical compositions of the present invention or fractions thereof typically comprise suitable pharmaceutically acceptable carriers, excipients, and vehicles selected based on the intended form of administration, and consistent with conventional pharmaceutical practices. Suitable pharmaceutical carriers, excipients, and vehicles are described in the standard text, Remington: The Science and Practice of Pharmacy (21^{st} Edition. 2005, University of the Sciences in Philadelphia (Editor), Mack Publishing Company), and in The United States Pharmacopeia: The National Formulary (USP 24 NF19) published in 1999.

In aspects of the invention, the compositions include without limitation at least one buffering agent or solution. Examples of buffering agents include, but are not limited to hydrochloric, hydrobromic, hydroiodic, sulfuric, phosphoric, formic, acetic, propionic, succinic, glycolic, glucoronic, maleic, furoic, citric, glutamic, benzoic, anthranilic, salicylic, phenylacetic, mandelic, embonic, pamoic, methanesulfonic, ethanesulfonic, pantothenic, benzenesulfonic, stearic, sulfanilic, algenic, galacturonic acid and mixtures thereof. Additional agents may also be included such as one or more of pregelatinized maize starch, polyvinyl pyrrolidone, hydroxypropyl methylcellulose, lactose, microcrystalline cellulose, calcium hydrogen phosphate, magnesium stearate, talc, silica, potato starch, sodium starch glycolate, sodium lauryl sulfate, sorbitol syrup, cellulose derivatives, hydrogenated edible fats, lecithin, acacia, almond oil, oily esters, ethyl alcohol, fractionated vegetable oils, methyl, propyl-p-hydroxybenzoates, sorbic acid and mixtures thereof. A buffering agent may additionally comprise one or more of dichlorodifluoromethane, trichloro fluoromethane, dichlorotetra fluoroethane, carbon dioxide, poly (N-vinyl pyrrolidone), poly (methylmethacrylate), polyactide, polyglycolide and mixtures thereof. In an embodiment, a buffering agent can be formulated as at least one medium including without limitation a suspension, solution, or emulsion. In other embodiments, a buffering agent may additionally comprise a formulatory agent including without limitation a pharmaceutically acceptable carrier, excipient, suspending agent, stabilizing agent or dispersing agent.

In aspects of the invention, a pharmaceutical composition is provided for oral administration of a scyllo-inositol compound and one or both of a valproic acid compound and lamotrigine for treatment of a bipolar disorder. In a particular aspect, a stable oral pharmaceutical composition for treatment of bipolar I disorder is provided comprising a substantially pure scyllo-inositol compound and one or both of a valproic acid compound and lamotrigine.

A composition for oral administration can be in the form of a capsule, sprinkle capsule tablet, or syrup. The active components can be combined with an oral, non-toxic pharmaceutically acceptable inert carrier such as lactose, starch, sucrose, methyl cellulose, magnesium stearate, glucose, calcium sulfate, dicalcium phosphate, mannitol, sorbital, and the like. For oral administration in a liquid form, the drug components may be combined with any oral, non-toxic, pharmaceutically acceptable inert carrier such as ethanol, glycerol, water, and the like. Suitable binders (e.g. gelatin, starch, corn sweeteners, natural sugars including glucose; natural and synthetic gums, and waxes), lubricants (e.g. sodium oleate, sodium stearate, magnesium stearate, sodium benzoate, sodium acetate, and sodium chloride), disintegrating agents (e.g. starch, methyl cellulose, agar, bentonite, and xanthan gum), flavoring agents, and coloring agents may also be combined in the compositions or components thereof. Compositions as described herein can further comprise wetting or emulsifying agents, or pH buffering agents.

In other aspects of the invention, a pharmaceutical composition is provided for parenteral administration of a scyllo-inositol compound and one or both of a valproic acid compound and lamotrigine. A parenteral formulation may include aqueous or non-aqueous solutions (e.g. water, isotonic saline, isotonic glucose solution, buffer solution, or other conventional solvents), syrups, aqueous or oil suspensions and emulsions with edible oil such as cottonseed oil, coconut oil, almond oil, or peanut oil. A composition intended for parenteral administration may also include conventional additives such as stabilizers, buffers, preservatives, or dispersing or suspending agents, for example, antioxidants such as methylhydroxybenzoate or similar additives. Dispersing or suspending agents that can be used for aqueous suspensions include synthetic or natural gums, such as tragacanth, alginate, acacia, dextran, sodium carboxymethylcellulose, gelatin, methylcellulose, and polyvinylpyrrolidone.

In aspects of compositions of the invention the ratio of a scyllo-inositol compound to one or both of a valproic acid compound and lamotrigine is selected to augment the activity of the scyllo-inositol compound or one or both of a valproic acid compound and lamotrigine. In particular aspects of the invention the ratio of a scyllo-inositol compound and one or both of a valproic acid compound and lamotrigine by weight is from about 1:1 to 1:110, 1:1 to 1:100, 1:1 to 1:75, 1:1 to 1:50, 1:1 to 1:25, 1:1 to 1:10, 1:1 to 1:5, and 1:1. In other aspects the ratio of one or both of a valproic acid compound and lamotrigine to a scyllo-inositol compound is from about 1:1 to 1:110, 1:1 to 1:100, 1:1 to 1:75, 1:1 to 1:50, 1:1 to 1:25, 1:1 to 1:10, 1:1 to 1:5, and 1:1.

This invention provides a conjugate comprising a scyllo-inositol compound linked to a valproic acid compound or lamotrigine. The invention also relates to isolated covalent conjugates of the invention, and compositions comprising covalent conjugates of the invention. Conjugates of a scyllo-inositol compound and a valproic acid compound or lamotrigine may be conjugated or linked with an intermediate spacer or linker. A suitable spacer or linker may be a mono- or disaccharide, an amino acid, a sulfate, a succinate, an acetate or an oligomeric polymeric spacer or linker comprising one or more of such moieties.

The invention also provides methods of preparing the above covalent conjugates that result in conjugates with improved pharmacokinetic properties, biological activity, and beneficial effects. The methods comprise incubating or reacting the scyllo-inositol compound with a valproic acid compound or lamotrigine under conditions that allow formation of a covalent linkage between the two compounds. The invention therefore contemplates a process for preparing a covalent conjugate comprising a scyllo-inositol compound covalently bonded or linked to a valproic acid compound or lamotrigine, the process comprising: incubating or reacting the scyllo-inositol compound with valproic acid compound or lamotrigine under suitable conditions (e.g. at an appropriate pH and sufficient time) for formation of a covalent bond or linkage between the scyllo-inositol and valproic acid compound or lamotrigine; and isolating the covalent conjugate. The above process for preparing a conjugate comprising a scyllo-inositol compound and a valproic acid compound or lamotrigine can provide a conjugate with a substantial amount of a scyllo-inositol compound covalently linked to a valproic acid compound or lamotrigine.

The invention further relates to a pharmaceutical formulation of a substantially pure covalent conjugate comprising a scyllo-inositol compound covalently linked to a valproic acid compound or lamotrigine. The formulation can provide beneficial effects, e.g. sustained beneficial effects compared to the scyllo-inositol compound or valproic acid compound or lamotrigine alone. In an embodiment, a pharmaceutical formulation is provided consisting essentially of covalent conjugates comprising a scyllo-inositol compound covalently linked without an intermediate spacer or linker to a valproic acid compound or lamotrigine. In another embodiment, a pharmaceutical formulation is provided consisting essentially of covalent conjugates comprising scyllo-inositol covalently linked with an intermediate spacer or linker to a valproic acid compound or lamotrigine.

Compounds, compositions or conjugates of the invention may be sterilized by, for example, filtration through a bacteria retaining filter, addition of sterilizing agents to the composition, irradiation of the composition, or heating the composition. Alternatively, the compounds, compositions or conjugates of the present invention may be provided as sterile solid preparations e.g. lyophilized powder, which are readily dissolved in sterile solvent immediately prior to use.

After the compounds, pharmaceutical compositions or conjugates have been prepared, they can be placed in an appropriate container and labeled for treatment of the indicated condition. Such labeling would include amount, frequency, and method of administration.

The invention also provides kits. In an aspect, the kit comprises a scyllo-inositol compound and one or both of a valproic acid compound and lamotrigine, or a pharmaceutical composition or conjugate of the invention. The kit can be a package which houses a container which contains the scyllo-inositol compound and one or both of a valproic acid compound and lamotrigine, or pharmaceutical composition or conjugate, and also houses instructions for administering the composition to a subject. A kit may contain a single dosage form or it may contain two or more dosage forms i.e. one for each compound to be administered. In an aspect, the kit comprises a fixed ratio dosage of a scyllo-inositol compound and one or both of a valproic acid compound and lamotrigine.

In aspects of the invention, a pharmaceutical pack or kit is provided comprising one or more containers filled with one or more of the ingredients of a pharmaceutical composition of the invention to provide a beneficial effect, in particular a sustained beneficial effect. Associated with such container(s) can be various written materials such as instructions for use, or a notice in the form prescribed by a governmental agency regulating the labeling, manufacture, use or sale of pharmaceuticals or biological products, which notice reflects approval by the agency of manufacture, use, or sale for human administration.

### Applications

The invention contemplates the use of a scyllo-inositol compound and one or both of a valproic acid compound and lamotrigine, a composition or conjugate of the invention for treating a bipolar disorder, in particular preventing and/or ameliorating disease severity, symptoms, and/or periodicity of recurrence of symptoms disclosed herein. The invention also contemplates treating a bipolar disorder in mammals using a combination of a scyllo-inositol compound, and one or both of a valproic acid compound and lamotrigine, compositions or conjugates of the invention. The present invention in embodiments may provide a composition comprising a scyllo-inositol compound, and one or both of a valproic acid compound and lamotrigine that provides beneficial effects for example, greater stability, efficacy, potency, and/or utility.

Greater efficacy and potency of a treatment of the invention in some aspects may improve the therapeutic ratio of treatment, reducing untoward side effects and toxicity. Selected methods of the invention may also improve long-standing symptoms even when treatment is begun long after the appearance of symptoms. Prolonged efficacious treatment may be achieved in accordance with the invention following administration of a scyllo-inositol and one or both of a valproic acid compound and lamotrigine, or composition of the invention.

In an embodiment, a combination of a scyllo-inositol compound and one or both of a valproic acid compound and lamotrigine can manifest as at least one of the following:
a) Prevention or reduction of manic episodes. In aspects of the invention, the combination induces at least about a 2%, 5%, 10%, 15%, 20%, 30%, 40%, 50%, 60%, 70%, 80%, or 90% decrease in manic episodes.
b) Prevention or reduction of depressive episodes. In aspects of the invention, the combination induces at least about a 2%, 5%, 10%, 15%, 20%, 30%, 40%, 50%, 60%, 70%, 80%, or 90% decrease in depressive episodes.
c) Prevention or reduction of mixed episodes. In aspects of the invention, the combination induces at least about a 2%, 5%, 10%, 15%, 20%, 30%, 40%, 50%, 60%, 70%, 80%, or 90% decrease in mixed episodes.
d) Extension of the time to recurrence of manic episodes, depressive episodes, and/or mixed episodes.
e) Reduction in suicidal thoughts and risk of suicide attempts and suicide deaths.
f) Improvement in social and/or occupational functioning.
g) Improvement in treatment resistance.

A scyllo-inositol compound and one or both of a valproic acid compound and lamotrigine, pharmaceutical compositions, conjugates and methods of the invention can be selected that have significant beneficial effects, in particular one or more significant beneficial effects of (a) through (g) above. A scyllo-inositol compound and one or both of a valproic acid compound and lamotrigine, pharmaceutical compositions, conjugates and methods of the invention can also be selected that have sustained beneficial effects. In an embodiment, a combination treatment or a pharmaceutical composition is provided with significant sustained beneficial effects, in particular sustained beneficial effects of one or more of (a) through (g) above, comprising a therapeutically effective amount of a scyllo-inositol compound and one or both of valproic acid compound and lamotrigine. In aspects of the invention, one or more of the beneficial effects provide enhanced therapeutic effects compared with conventional treatments.

The invention contemplates compositions comprising a scyllo-inositol compound and one or both of a valproic acid compound and lamotrigine in combination with other therapeutics. In an aspect of the invention, the other therapeutics include, without limitation, risperidone, lithium, aripiprazole, quetiapine, olanzapine, asenapine, arisulpride, ziprasidone, carbamazepine, oxcarbazepine, paliperidone, fluoxetine, benzodiazepine, or combinations thereof. In embodiments of the invention, the other therapeutics include, without limitation, risperidone, lithium, olanzapine, and carbamazepine or combinations thereof. In embodiments of the invention, the other therapeutic is risperidone, lithium or olanzapine, or combinations thereof. In embodiments of the invention, the other therapeutic is risperidone or olanzapine. A combination therapy of the invention optionally with one or more additional therapeutic may provide an additive or synergistic effect, in particular synergistic effect.

In an aspect the invention provides the use of a scyllo-inositol compound, and one or both of a valproic acid compound and lamotrigine, or a composition comprising a scyllo-inositol compound and one or both of a valproic acid compound and lamotrigine, for the preparation of a medicament for treatment, prolonged or sustained treatment or maintenance therapy of a bipolar disorder. In a further aspect the invention provides the use of a scyllo-inositol compound, and one or both of a valproic acid compound and lamotrigine, or a composition comprising a scyllo-inositol compound and one or both of a valproic acid compound and lamotrigine, for preparation of a pharmaceutical composition to be employed through oral administration for treatment of a bipolar disorder.

### Administration

Scyllo-inositol compound, one or both of a valproic acid compound and lamotrigine, conjugates, and compositions of the present invention can be administered by any means that produce contact of the active agent(s) with the agent's sites of action in the body of a subject or patient to produce a therapeutic effect, in particular a beneficial effect, in particular a sustained beneficial effect. The active ingredients can be administered simultaneously or sequentially and in any order at different points in time to provide the desired beneficial effects. A scyllo-inositol compound and one or both of a valproic acid compound and lamotrigine, a conjugate and/or a composition of the invention can be formulated for sustained release, for delivery locally or systemically. It lies within the capability of a skilled physician to select a form and route of administration that optimizes the effects of the treatments of the present invention to provide desired therapeutic effects, in particular beneficial effects, more particularly sustained beneficial effects.

A scyllo-inositol compound and one or both of a valproic acid compound and lamotrigine, conjugates, and/or compositions may be administered in oral dosage forms such as tablets, capsules (each of which includes sustained release or timed release formulations), sprinkle capsules, pills, powders, granules, elixirs, tinctures, suspensions, syrups, and emulsions. They may also be administered in intravenous (bolus or infusion), intraperitoneal, subcutaneous, or intramuscular forms, all utilizing dosage forms well known to those of ordinary skill in the pharmaceutical arts. A scyllo-inositol compound and one or both of a valproic acid compound and lamotrigine, conjugates, and/or compositions may be administered by intranasal route via topical use of suitable intranasal vehicles, or via a transdermal route, for example using conventional transdermal skin patches. A sustained release or extended release formulation can also be used for the therapeutic agents.

In aspects of the invention a scyllo-inositol compound and one or both of a valproic acid compound and lamotrigine, conjugates, and/or compositions are administered by peripheral administration, in particular by intravenous administration, intraperitoneal administration, subcutaneous administration, intramuscular administration, oral administration, topical administration, transmucosal administration, or pulmonary administration.

An effective amount of a scyllo-inositol compound and one or both of a valproic acid compound and lamotrigine, conjugate, and/or composition which will be effective in the treatment of a bipolar disorder to provide desired effects, in particular beneficial effects, more particularly sustained beneficial effects, will depend on the nature of the disease, and can be determined by standard clinical techniques. The precise dose to be employed in the formulation will also depend on the route of administration, and the seriousness of the disease, and should be decided according to the judgment of the practitioner and each patient's circumstances. In particular, the dosage regimen of the invention will vary depending upon known factors such as the mode and route of administration; the species, age, sex, health, medical condition, and weight of the patient, the nature and extent of the symptoms, the kind of concurrent treatment, the frequency of treatment, the renal and hepatic function of the patient, and the desired effect.

In an aspect, the scyllo-compound is scyllo-inositol and the effective amount of scyllo-inositol is the amount required to reduce the level of myo-inositol in a patient's brain. In a particular embodiment, an effective amount of scyllo-inositol is the amount required to reduce the level of myo-inositol in a patient's brain to less than 60% from a baseline measurement prior to administration. In a particular embodiment, an effective amount of scyllo-inositol is the amount required to reduce the level of myo-inositol in a patient's brain by about 20% to about 55%, about 20% to about 50%, about 25% to about 45%, or about 25% to about 35% from a baseline measurement prior to administration. In an embodiment, an effective amount of scyllo-inositol is the amount required to reduce the level of myo-inositol in a patient's brain by about 20% to about 50% from a baseline measurement prior to administration.

In a particular embodiment, the amount of scyllo-inositol administered is about 1 to about 5000 mg/day. In a particular embodiment, the amount of scyllo-inositol administered is about 10 to about 2500 mg/day. In a particular embodiment, the amount of scyllo-inositol administered is about 100 to about 2,500 mg per day. In a particular embodiment, the amount of scyllo-inositol administered is about 150 to about 3,000 mg per day. In a particular embodiment, the amount of scyllo-inositol administered is about 200 to about 2,000 mg per day. In a particular embodiment, the amount of scyllo-inositol administered is about 500 to about 1,500 mg per day. In a particular embodiment, the amount of scyllo-inositol administered is about 750 to about 1,250 mg per day. In a particular embodiment, the amount of scyllo-inositol administered is about about 1,000 mg per day. In a particular embodiment, the amount of scyllo-inositol administered is 1,000 mg per day. In a particular embodiment, the amount of scyllo-inositol administered is about 700 to about 1200 mg per day. In a particular embodiment, the amount of scyllo-inositol administered is about 800 to about 1100 mg per day. In a particular embodiment, the amount of scyllo-inositol administered is about 900 to about 1100 mg per day. In a particular embodiment, the amount of scyllo-inositol administered is about 500 mg per day. In a particular embodiment, the amount of scyllo-inositol administered is 500 mg per day. In a particular embodiment, the foregoing amounts of scyllo-inositol are administered once daily. In a particular embodiment, the foregoing amounts of scyllo-inositol are administered twice daily (BID). In a particular embodiment, the amount of scyllo-inositol administered is about 500 mg twice daily. In a particular embodiment, the amount of scyllo-inositol administered is 250 mg two times daily. In a particular embodiment, the amount of scyllo-inositol administered is 250 mg four times daily. In a particular embodiment, the amount of scyllo-inositol administered is 500 mg twice daily. In another particular embodiment, the foregoing amounts of scyllo-inositol are administered three times daily.

In an embodiment, an effective amount of a valproic acid compound is between about 50 and 20,000 mg. In an embodiment, an effective amount of valproic acid compound is between about 100 and 10,000 mg. In an embodiment, an effective amount of a valproic acid compound is between about 750 and 5,000 mg per day. In an embodiment, an effective amount of valproic acid is between about 1,500 and 2,500 mg per day. In an embodiment, an effective amount of valproic acid is between 1,500 and 2,500 mg per day. In an embodiment, an effective amount of a valproic acid compound is 60 mg/kg/day. In an embodiment, an effective amount of a valproic acid compound is less than 750 mg per day. In an embodiment, an effective amount of a valproic acid compound is less than 625 mg per day. In an embodiment, an effective amount of a valproic acid compound is less than or equal to 500 mg per day. In an embodiment, an effective amount of a valproic acid compound is less than or equal to 250 mg per day. In an embodiment, an effective amount of a valproic acid compound is less than 60 mg/kg/day. In an embodiment, an effective amount of a valproic acid compound is less than or equal to 50 mg/kg/day. In an embodiment, an effective amount of a valproic acid compound is less than or equal to 40 mg/kg/day.

In an embodiment, an effective amount in a dosage form of valproic acid compound is 125 mg. In an embodiment, an effective amount in a dosage form of a valproic acid compound is 250 mg. In an embodiment, an effective amount in a dosage form of a valproic acid compound is 500 mg. In an embodiment, an effective amount of a valproic acid compound in a delayed release tablet is 125, 250 or 500 mg. In an embodiment, an effective amount of a valproic acid compound in an extended release tablet is 250 mg and 500 mg. In an embodiment, an effective amount of a valproic acid compound in a sprinkle capsule is 125 mg. In an embodiment, an effective amount of a valproic acid compound in capsules is 250 mg. In an embodiment, an effective amount of a valproic acid compound in a liquid dosage form is 50 mg/5 ml to 500 mg/ml. In an embodiment, an effective amount of a valproic acid compound in a syrup is 250 mg/ml. In an embodiment, an effective amount of a valproic acid compound in an injectable dosage form is 100 mg/ml.

In an embodiment, the amount of valproic acid administered to a subject is that which achieves a plasma concentration in the subject of about 1 to 500 μg/mL. In an embodiment, the amount of valproic acid administered to a subject is that which achieves a plasma concentration in the subject of about 10 to 250 μg/mL. In an embodiment, the amount of valproic acid administered to a subject is that which achieves a plasma concentration in the subject of about 50 to 125 μg/mL. In an embodiment, the amount of valproic acid administered to a subject is that which achieves a plasma concentration in the subject between 50 and 125 μg/mL.

In an embodiment, an effective amount of lamotrigine is about 25 mg, 50 mg, 100 mg, 150 mg and 200 mg (e.g. scored tablets), or 2 mg, 5 mg and 25 mg (e.g. chewable dispersible tablets). In particular embodiments, the amount of lamotrigine is 25 mg for administration to patients taking a valproic acid compound. In particular embodiments, the amount of lamotrigine is 25 mg or 500 mg for patients not taking a valproic acid compound. In particular embodiments, an effective amount of lamotrigine is 25 mg per day for weeks one and two, 50 mg per day taken in two divided doses for weeks three and four, and 25 to 50 mg thereafter. In an embodiment, an effective amount is between 50 and 500 mg per day taken in two divided doses. In an embodiment, an effective amount of lamotrigine is 200 mg per day.

In an embodiment, the amount of lamotrigine administered to a subject is that which achieves a plasma concentration in the subject of about 0.1 to 1,000 μg,/mL. In an embodiment, the amount of lamotrigine administered to a subject is that which achieves a plasma concentration in the subject of about 1 to 500 μmol/L. In an embodiment, the amount of lamotrigine administered to a subject is that which achieves a plasma concentration in the subject of about 10 to 50 μmol/L. In an embodiment, the amount of lamotrigine administered to a subject is that which achieves a plasma concentration in the subject of 10 to 50 μmol/L.

In an aspect of the invention, a method of treating a bipolar disorder in a subject is provided comprising administering 1,000 mg per day or 500 mg twice a day of scyllo-inositol and 1,500 to 2,500 mg per day of a valproic acid compound. In an aspect of the invention, a method of reducing manic episodes in a subject suffering from bipolar disorder is provided comprising administering 1,000 mg per day or 500 mg twice a day of scyllo-inositol and 1,500 to 2,500 mg per day of a valproic acid compound.

In an aspect of the invention, a method of treating a bipolar disorder is provided comprising administering 1,000 mg per day or 500 mg twice a day of scyllo-inositol and 200 mg per day of lamotrigine. In an aspect of the invention, a method of reducing manic episodes in a subject suffering from bipolar disorder is provided administering 1,000 mg per day or 500 mg twice a day of scyllo-inositol and 200 mg per day of lamotrigine.

The combined administration of a scyllo-inositol compound and one or both of valproic acid compound and lamotrigine(s) may require less of the generally-prescribed dose for any of the agents when used alone and or may result in less frequent administration of either, both or all agents. In aspects of the invention valproic acid compound and/or lamotrigine are present in doses that are at least about 1.1 to 1.4, 1.5, 2, 3, 4, 5, 6, 7, 8, 9, or 10 fold lower than the doses of each compound alone required to treat a bipolar disorder.

A composition or treatment of the invention may comprise a unit dosage of a scyllo-inositol compound and at least one of one or both of a valproic acid compound and lamotrigine. A “unit dosage” or “dosage unit” refers to a unitary i.e., a single dose which is capable of being administered to a patient, and which may be readily handled and packed, remaining as a physically and chemically stable unit dose comprising either the active agents as such or a mixture with one or more solid or liquid pharmaceutical excipients, carriers, or vehicles.

A subject may be treated with a scyllo-inositol compound and one or both of a valproic acid compound and lamotrigine, or a conjugate or composition of the invention on substantially any desired schedule. A scyllo-inositol compound (e.g. scyllo-inositol) and one or both of a valproic acid compound and lamotrigine, or a conjugate or composition of the invention may be administered one or more times per day, in particular 1 or 2 times per day, 1, 2, 3, 4, 5 or more times per week, 1 to 20, 1 to 15, 1 to 10 or 1 to 5 times a month or continuously. However, a subject may be treated less frequently, such as every other day or once a week, or more frequently.

A scyllo-inositol compound and one or both of a valproic acid compound and lamotrigine, or a conjugate or composition of the invention may be administered to a subject for about or at least about 1 week, 2 weeks to 4 weeks, 2 weeks to 6 weeks, 2 weeks to 8 weeks, 2 weeks to 10 weeks, 2 weeks to 12 weeks, 2 weeks to 14 weeks, 2 weeks to 16 weeks, 2 weeks to 6 months, 2 weeks to 12 months, 2 weeks to 18 months, or 2 weeks to 24 months, or more than 24 months, periodically or continuously.

In a combination therapy to treat a bipolar disorder, a scyllo-inositol compound compound and one or both of valproic acid compound and lamotrigine can be administered simultaneously or at separate intervals. When administered simultaneously the scyllo-inositol compound and one or both of a valproic acid compound and lamotrigine can be incorporated into a single pharmaceutical composition, e.g., a pharmaceutical combination therapy composition. Alternatively, two or more separate compositions, i.e., one containing the scyllo-inositol compound compound(s) and the other(s) containing the valproic acid compound and/or lamotrigine(s), can be administered simultaneously.

When separately administered, therapeutically effective amounts of compositions containing a scyllo-inositol compound compound, and valproic acid compound and/or lamotrigine(s) are administered on a different schedule. One may be administered before the other as long as the time between the administrations falls within a therapeutically effective interval. A therapeutically effective interval is a period of time beginning when one of either the (a) scyllo-inositol compound, in particular, the scyllo-inositol, or (b) valproic acid compound and/or lamotrigine is (are) administered to a mammal and ending at the limit of the desired effect in the treatment of the bipolar disorder from the combination of (a) and (b).

The invention will be described in greater detail by way of a specific example. The following example is offered for illustrative purposes, and is not intended to limit the invention in any manner. Those of skill in the art will readily recognize a variety of noncritical parameters which can be changed or modified to yield essentially the same results.

### EXAMPLE

A Phase 2, placebo-controlled, safety and efficacy study of oral ELND005 (oral scyllo-inositol) as an adjunctive maintenance treatment in patients with Bipolar I Disorder (BPD 1) to delay the time to occurrence of mood episodes is being conducted. Study patients (18 to 65 years) are administered Lamotrigine (maximum 200 mg/day, 10-50 μmol/L plasma concentration) or Valproic acid (1500-2500 mg/day, 50-125 μg/mL plasma concentration) and ELND005 (500 mg twice/day) or Lamotrigine (maximum 200 mg/day, 10-50 μmol/L plasma concentration) or Valproic acid (1500-2500 mg/day, 50-125 μg/mL plasma concentration) and a placebo.

The following are the inclusion criteria for the study:
i. Meets the DSM-IV-TR criteria for BPD I by the Structural Clinical Interview for DSM Disorders (SCID), prior to the Screening Visit.
ii. Has a history in the last 3 years of ≧1 manic or mixed episodes of sufficient severity that required hospitalization and/or treatment with a mood stabilizer or antipsychotic, or confirmed by a family member or medical records to ensure the episode fulfills the DSM-IV-TR criteria.
iii. Has experienced a mood episode of any polarity within 90 days prior to the Screening Visit and responded to StOC therapy.
iv. Is euthymic at the Screening Visit (ie, score of ≦12 on the Montgomery-Åsberg Depression Rating Scale (MADRS) and a score of ≦12 on the Y-MRS).
v. Is receiving maintenance treatment for his or her BPD I with either LTG or VPA; on stable doses for past 4 weeks and therapeutic drug levels (total VPA 50-125 μg/mL and LTG 10-50 μmol/L or as deemed appropriate by the investigator). Dose adjustments made for tolerability reasons will be acceptable.

A study patient must meet the following additional criteria to be eligible for randomization in the Double-blind Randomization Phase of this study: Maintained in a stable euthymic state during Phase 1, defined as Young Mania Rating Scale (Y-MRS) and MADRS scores of ≦12, with the following exceptions: a maximum of 2 nonconsecutive excursions are allowed throughout Phase 1. Excursions are defined as Y-MRS or MADRS scores >12 but ≦15.

The following are the exclusion criteria for the study:
i. Woman of childbearing potential who is unwilling or unable to use an acceptable method of birth control or is using a prohibited contraceptive method.
ii. Is found to be actively suicidal on the Columbia-Suicide Severity Rating Scale (C-SSRS) (answer of “yes” to question 4 or 5 [current or over the last 30 days]) or a score of ≧4 on the MADRS item 10 at the Screening Visit.
iii. Has suboptimally treated thyroid disease as evidenced by thyroid-stimulating hormone (TSH) >3 mIU/L at the Screening Visit.
iv. Has received electroconvulsive therapy (ECT) during the current episode or within 6 months prior to the Screening Visit.
v. Has an estimated glomerular filtration rate <40 mL/min/1.73 m2 according to the Modification of Diet in Renal Disease formula.

A study patient who meets any of the above and any of the criteria below will not be eligible for enrollment in the Double-blind Randomization Phase of this study:
i. Has current signs or symptoms of psychosis.
ii. Has become actively suicidal as defined by C-SSRS answer of “yes” to question 4 or 5 (current or over the last 30 days) and/or has a score of ≧4 on MADRS item 10

The primary outcome is the time to recurrence of any mood episode. The secondary outcomes include the following:
i. Proportion of study participants with recurrence of any mood episode.
ii. Time to recurrence of a depressive episode.
iii. Time to recurrence of a manic/hypomanic or a mixed episode.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the methodologies etc. which are reported therein which might be used in connection with the invention. Nothing herein is to be construed as an admission that the invention is not entitled to antedate such disclosure by virtue of prior invention.

What is claimed is:

1. A method of delaying the occurrence of a mood episode in a subject having bipolar disorder comprising administering to said subject effective amounts of scyllo-inositol and one or both of valproic acid and lamotrigine.

2. A method of reducing the level of myo-inositol in the brain of a subject comprising administering to the subject effective amounts of scyllo-inositol and one or both of valproic acid and lamotrigine.

3. A method for treating a bipolar disorder comprising administering to the subject a combination of an effective amount of scyllo-inositol and an effective amount of one or both of a valproic acid compound and lamotrigine.

4. The method as claimed in claim 3 wherein the bipolar disorder is bipolar I disorder.

5. The method as claimed in any one of claims 1 to 3 wherein the effective amounts of scyllo-inositol and one or both of valproic acid and lamotrigine are combined and administered to the subject in a single formulation.

6. The method as claimed in any one of claims 1 to 3 wherein the effective amounts of the scyllo-inositol compound and one or both of a valproic acid compound and lamotrigine are administered to the subject sequentially.

7. The method as claimed in any one of claims 1 to 3, wherein the subject is administered an effective amount of scyllo-inositol and an effective amount of valproic acid and not lamotragine.

8. The method as claimed in claim 7, wherein valproic acid is administered to the subject prior to scyllo-inositol.

9. The method as claimed in claim 8, wherein the concentration of valproic acid in the subject's plasma is about 1 to 500 μg/mL.

10. The method as claimed in claim 8, wherein the concentration of valproic acid in the subject's plasma is about 50 to 125 μg/mL.

11. The method as claimed in claim 8, wherein the concentration of valproic acid in the subject's plasma is between 50 and 125 μg/mL.

12. The method as claimed in claim 6, wherein the combination administered to the subject is an effective amount of scyllo-inositol and an effective amount of lamotrigine and not valproic acid.

13. The method as claimed in claim 12, wherein lamotrigine is administered to the subject prior to scyllo-inositol.

14. The method as claimed in claim 13, wherein the concentration of lamotrigine in the subject's plasma is about 1 to 500 μg/mL.

15. The method as claimed in claim 13, wherein the concentration of lamotrigine in the subject's plasma is about 10 to 50 μg/mL.

16. The method as claimed in claim 13, wherein the concentration of lamotrigine in the subject's plasma is between 10 and 50 μg/mL.

17. The method as claimed in any one of claims 1 to 3, wherein the amount of scyllo-inositol administered to the subject is from 1 to 5,000 mg/day.

18. The method as claimed in claim 17 wherein the amount of scyllo-inositol administered to the subject is from 100 to 2,000 mg/day.

19. The method as claimed in claim 17 wherein the amount of scyllo-inositol administered to the subject is 1,000 mg/day.

20. The method as claimed in claim 17 wherein the amount of scyllo-inositol administered to the subject is 500 mg BID.

21. The method as claimed in claim 12 wherein the amount of scyllo-inositol administered is 500 mg BID and the concentration of valproic acid in the subject's plasma is is between 50 and 125 μg/mL.

22. The method as claimed in claim 17 wherein the amount of scyllo-inositol administered is 500 mg BID and the concentration of lamotrigine in the subject's plasma is between 10 and 50 μg/mL.

23. The method as claimed in claim 1 wherein the effective amounts of the scyllo-inositol compound and one or both of a valproic acid compound and lamotrigine are synergistically effective amounts.

24. A pharmaceutical composition for treating a bipolar disorder comprising a scyllo-inositol compound and one or both of a valproic acid compound and lamotrigine.

25. The pharmaceutical composition according to claim 24 comprising about 100 to about 1500 mg of the scyllo-inositol compound.

26. Use of a scyllo-inositol compound and one or both of a valproic acid compound and lamotrigine for the treatment of a bipolar disorder.

27. A kit comprising a scyllo-inositol compound and one or both of a valproic acid compound and lamotrigine, a container, and instructions for use in the treatment of a bipolar disorder in a subject.
